(19) United States
(12) Patent Application Publication
(10) Pub. No.: US 2026/0191784 A1
(43) Pub. Date: Jul. 9, 2026

(54) METHOD FOR INDUSTRIAL PRODUCTION OF SPRAY-DRIED PIPERACILLIN-TAZOBACTAM POWDER

(71) Applicants: Kim Eng SIM, Nanjin, Jiangsu (CN); Nanjing Pharmacare Co., Ltd., Nanjing, Jiangsu (CN)

(72) Inventors: Kim Eng SIM, Nanjing, Jiangsu (CN); Jian LIANG, Nanjing, Jiangsu (CN); Fang CAO, Nanjing, Jiangsu (CN); Jiacheng LIANG, Nanjing, Jiangsu (CN); Ying YU, Nanjing, Jiangsu (CN)

(73) Assignee: Nanjing Pharmacare Co., Ltd., Nanjing, Jiangsu (CN)

(21) Appl. No.: 19/126,006

(22) PCT Filed: Oct. 31, 2022
(86) PCT No.: PCT/CN2022/128620

§ 371 Date: Apr. 30, 2025

(51) Int. Cl. A61K 9/16; A61K 31/431; A61K 31/496

(52) U.S. Cl. CPC A61K 9/1641; A61K 9/1611; A61K 9/1688; A61K 9/1694; A61K 31/431; A61K 31/496

(57) ABSTRACT

Provided is a method for industrial production of spray-dried piperacillin-tazobactam powder, comprising the following steps: 1) preparing a mixed solution of piperacillin or a pharmaceutically acceptable salt thereof and tazobactam or a pharmaceutically acceptable salt thereof, and maintaining the pH value at 6.0-7.5; 2) adding a stabilizer into the solution obtained in the step 1) to give a mixed solution, wherein the stabilizer is selected from one or more of a carbohydrate, an amino acid, and a polyol; and 3) subjecting the mixed solution obtained in the step 2) to spray drying to give the spray-dried piperacillin-tazobactam powder.

### FIELD OF THE INVENTION

The invention belongs to the field of medicine and relates to a method for industrial production of piperacillin and tazobactam spray-dried powder.

### BACKGROUND OF THE INVENTION

Piperacillin and tazobactam is a combination drug for the treatment or control of bacterial infections by parenteral administration, and its active ingredients comprise piperacillin or a salt thereof and tazobactam or a salt thereof. Piperacillin is a β-lactam antibiotic, and its β-lactam ring is liable to be destroyed by β-lactam enzyme and lose its antibacterial activity. Tazobactam is a β-lactamase inhibitor, which can reduce the destruction of piperacillin by β-lactamase producing bacteria and protect the anti-bacterial activity of piperacillin.

The indications of Piperacillin and tazobactam include: intra-abdominal infections, such as appendicitis (complicated by rupture or abscess) and peritonitis caused by Escherichia coli; nosocomial pneumonia caused by Staphylococcus aureus or the like; infections of skin and skin tissue, such as cellulitis, cutaneous abscesses and ischemic/diabetic foot infections caused by Staphylococcus aureus; gynecological infections, such as postpartum endometritis or pelvic inflammatory disease caused by Escherichia coli; and community-acquired pneumonia caused by Haemophilus influenzae. The drug products of piperacillin and tazobactam currently available on the market are mainly classified into two types, one is a premixed frozen bag stored at −20° C., and the other is a lyophilized powder for injection packaged in a vial stored at the room temperature. Premixed frozen bags require −20° C. cold-chain transportation and storage, increasing the cost and price of drugs. It needs to be thawed in advance at the room temperature or under refrigeration before using, which is time-consuming and labor-intensive. Lyophilized powder for injection packaged in vials are produced by using freeze-drying process, which has long production cycle, small production capacity, large energy consumption and large loss.

Spray drying is a mature technology for powder production. Its principle is to spray the material liquid into a fine mist in a hot gas stream to increase the contact area between the liquid and the gas phase, and dry it directly into fine powder. Spray drying is widely used in food production, such as milk powder production. However, its application in production of drugs is still very limited for an obvious reason that the heat sensitive materials in drugs may be degraded by the high temperature during the spray drying process. Although a few patents have disclosed the application of spray drying methods to the preparation of drugs (Patent Literatures 1-3), no industrial production of piperacillin and tazobactam by using spray drying methods has been reported. Piperacillin and tazobactam is known to be unstable in an aqueous solution. On the one hand, piperacillin is α-lactam antibiotic, which is liable to be decomposed by high temperature, acid or base, oxidant, metal ions or the like after being dissolved in water, and generate antigenic degradation products, resulting in allergic reactions. On the other hand, with the degradation of piperacillin, the pH value of the solution is decreased, and the acid-base balance of piperacillin and tazobactam is shifted to the acid form, resulting in crystallization of piperacillin and tazobactam and formation of insoluble piperacillin and tazobactam particles.

Therefore, there is still much room for improvement in the industrial production of piperacillin and tazobactam in the existing technology, and there is an urgent desire to develop an industrial production method that can produce high purity and stability piperacillin and tazobactam with high efficiency and high yield.

### PRIOR ART LITERATURES

### Patent Literatures

Patent Literature 1: U.S. Pat. No. 6,001,800
Patent Literature 2: U.S. Pat. No. 6,479,049
Patent Literature 3: WO2014/139329

### SUMMARY OF THE INVENTION

The technical problem to be solved by this invention is to provide an industrial production method that can produce high purity and stability piperacillin and tazobactam with high efficiency and high yield.

In order to solve the above technical problem, the present inventors carried out thorough research. As a result, the inventors surprisingly found that the method of spray drying can produce high purity piperacillin and tazobactam powder with high efficiency and high yield, thus completing the present invention.

In particular, the present invention provides the following method for industrial production of piperacillin and tazobactam spray-dried powder.

1. A method for industrial production of piperacillin and tazobactam spray-dried powder, comprising the steps of: (1) preparing a mixture solution of piperacillin or a pharmaceutically acceptable salt thereof and tazobactam or a pharmaceutically acceptable salt thereof while maintaining the pH value at 6.0~7.5; (2) adding a stabilizer to the solution obtained in the step (1) to give a mixed solution, wherein the stabilizer is one or more selected from the group consisting of carbohydrates, amino acids and polyols; and (3) spray drying the mixed solution obtained in the step (2) to give the piperacillin and tazobactam spray-dried powder.

2. The method for industrial production of piperacillin and tazobactam spray-dried powder according to item 1, wherein, in the mixture solution prepared by the step (1), the ratio of the content of piperacillin sodium or a pharmaceutically acceptable salt thereof calculated as piperacillin free acid to the content of tazobactam or a pharmaceutically acceptable salt thereof calculated as tazobactam free acid is approximately 8:1.

3. The method for industrial production of piperacillin and tazobactam spray-dried powder according to item 1 or 2, wherein, in the step (1), a powder mixture of piperacillin or a pharmaceutically acceptable salt thereof and tazobactam or a pharmaceutically acceptable salt thereof is dissolved in water to prepare the mixture solution; alternatively, piperacillin or a pharmaceutically acceptable salt thereof and t tazobactam or a pharmaceutically acceptable salt thereof are dissolved in water, separately, and then mixed to prepare the mixture solution; alternatively, piperacillin acid and/or tazobactam acid is converted into a salt with sodium bicarbonate, sodium carbonate or sodium hydroxide to prepare a solution of piperacillin sodium and/or tazobactam sodium, respectively, and then formulated into the mixture solution,

4. The method for industrial production of piperacillin and tazobactam spray-dried powder according to item 1 or 2, wherein, the concentration calculated as piperacillin of the mixed solution obtained in the step (2) is 1~50% by weight.

5. The method for industrial production of piperacillin and tazobactam spray-dried powder according to item 1 or 2, wherein, the concentration calculated as piperacillin of the mixed solution obtained in the step (2) is 26~38% by weight.

6. The method for industrial production of piperacillin and tazobactam spray-dried powder according to item 1 or 2, wherein, the stabilizer added in the step (2) is a polyol.

7. The method for industrial production of piperacillin and tazobactam spray-dried powder according to item 6, wherein, the stabilizer is polyethylene glycol or glycerol.

8. The method for industrial production of piperacillin and tazobactam spray-dried powder according to item 6, wherein, the stabilizer is polyethylene glycol 200~600.

9. The method for industrial production of piperacillin and tazobactam spray-dried powder according to item 6, wherein, the stabilizer is polyethylene glycol 300~400.

10. The method for industrial production of piperacillin and tazobactam spray-dried powder according to item 1 or 2, wherein, in the step (2), 0~50 parts by weight of the stabilizer is added based on 100 parts by weight of piperacillin.

11. The method for industrial production of piperacillin and tazobactam spray-dried powder according to item 1 or 2, wherein, in the step (2), 0~20 parts by weight of the stabilizer is added based on 100 parts by weight of piperacillin. 12. The method for industrial production of piperacillin and tazobactam spray-dried powder according to item 1 or 2, wherein, in the step (2), 2.5~15 parts by weight of the stabilizer is added based on 100 parts by weight of piperacillin.

13. The method for industrial production of piperacillin and tazobactam spray-dried powder according to item 1 or 2, wherein, in the step (2), an aminocarboxylic acid chelating agent EDTA is further added to the solution obtained in the step (1).

14. The method for industrial production of piperacillin and tazobactam spray-dried powder according to item 1 or 2, wherein, the mixed solution obtained in the step (2) is stored at a storage temperature of −12~8° C.

15. The method for industrial production of piperacillin and tazobactam spray-dried powder according to item 1 or 2, wherein, the mixed solution obtained in the step (2) is stored at a storage temperature of −10~0° C.

16. The method for industrial production of piperacillin and tazobactam spray-dried powder according to item 1 or 2, wherein, in the step (3), the spray drying is carried out at an inlet air temperature of 160~240° C.

17. The method for industrial production of piperacillin and tazobactam spray-dried powder according to item 1 or 2, wherein, in the step (3), the spray drying is carried out at an inlet air temperature of 185~200° C.

18. The method for industrial production of piperacillin and tazobactam spray-dried powder according to item 1 or 2, wherein, in the step (3), the spray drying is carried out at an outlet air temperature of 80~130° C.

19. The method for industrial production of piperacillin and tazobactam spray-dried powder according to item 1 or 2, wherein, in the step (3), the spray drying is carried out at an outlet air temperature of 100~115° C.

### Effect of Invention

According to the method for industrial production of piperacillin and tazobactam spray-dried powder of the present invention, a high purity and high stability piperacillin and tazobactam powder can be produced with high efficiency and high yield. The invention has a high application value in the pharmaceutical field.

### DESCRIPTION OF THE FIGURES

FIG. 1: Morphology of piperacillin and tazobactam spray-dried powder under an optical microscope.

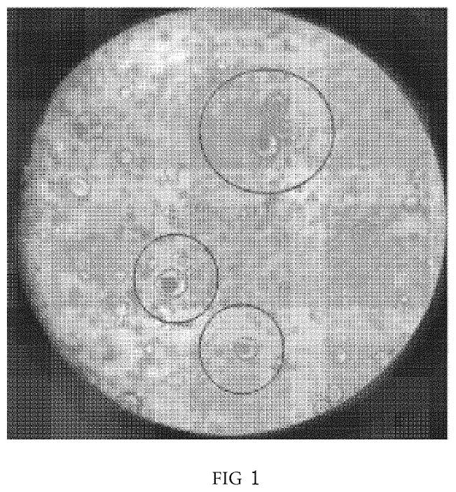

### DETAILED DESCRIPTION OF THE INVENTION

As mentioned above, the drug products of piperacillin and tazobactam currently available on the market are mainly classified into two types, one is a premixed frozen bag stored at −20° C., and the other is a lyophilized powder for injection packaged in a vial stored at the room temperature. However, there have been no reports of industrial production using spray drying method. As piperacillin is unstable in an aqueous solution, and liable to be decomposed by high temperature, acid or base, oxidizer, metal ions or the like after being dissolved in water, there are problems in the stability of an aqueous solution during storage. It is also concerned that piperacillin and tazobactam may be degraded by the high temperature during spray drying.

The present inventors have attempted to prepare piperacillin and tazobactam powder by a method of spray drying. As a result of the research, the inventors found that in the preparation of piperacillin and tazobactam powder by a method of spray drying, the stability of piperacillin and tazobactam can be increased by adding a stabilizer (such as carbohydrates, amino acids and/or polyols) to the formulation. Accordingly, piperacillin and tazobactam powder of high purity can be produced with high efficiency and high yield. At the same time, by reducing the storage temperature of piperacillin and tazobactam solution, the stability of piperacillin and tazobactam solution can be significantly improved and the generation of impurities can be reduced. In addition, the use of a citrate or the like as a pH buffering agent to maintain the pH value of a solution of piperacillin and tazobactam at 6.0~7.5 can confer a better stability to piperacillin and tazobactam. EDTA as a chelating agent of metal ions also can further increase the stability of piperacillin and tazobactam.

Based on the above research, the present invention provides a method for industrial production of piperacillin and tazobactam spray-dried powder with high purity, low impurity, high efficiency and high yield. The method includes: preparing a solution of piperacillin and tazobactam, adding a stabilizer of carbohydrate, amino acid and/or polyols, adding EDTA and sodium citrate as required to obtain a mixed solution, performing a storage at a low temperature condition for ensuring purity and reduced impurities, performing a sterilization filtration or performing no sterilization filtration, then performing the spray drying, and obtaining a sterile or non-sterile active pharmaceutical ingredient (API) of piperacillin and tazobactam. The sterile API is dispensed under an aseptic condition to produce the final drug product.

The method for industrial production of the present invention specifically comprises the following steps:

(1) Preparing a mixture solution of piperacillin or a pharmaceutically acceptable salt thereof and tazobactam or a pharmaceutically acceptable salt thereof.

In this step, the pH is maintained in a range of 6.0~7.5, for example, 6.0~7.0, 6.1~6.9, 6.2~6.8, 6.3~6.7, 6.4~6.6, preferably at around 6.5. By maintaining the pH in this range, piperacillin and tazobactam have a better stability. The pH can be adjusted using methods known in the field, such as the addition of citrate or the like.

In this step, a mixture of piperacillin or a pharmaceutically acceptable salt thereof and tazobactam or a pharmaceutically acceptable salt thereof may be dissolved in water to prepare the mixture solution. Alternatively, piperacillin or a pharmaceutically acceptable salt thereof and tazobactam or a pharmaceutically acceptable salt thereof may be dissolved in water, separately, and then mixed to prepare the mixture solution. Alternatively, piperacillin acid and/or tazobactam acid may be converted into a salt with sodium bicarbonate, sodium carbonate or sodium hydroxide to prepare a solution of piperacillin sodium and/or tazobactam sodium, respectively, and then formulated into the mixture solution. If piperacillin acid and/or tazobactam acid are converted into a salt with sodium bicarbonate, sodium carbonate or sodium hydroxide to prepare a solution of piperacillin sodium and/or tazobactam sodium, respectively, and then formulated into the mixture solution, the degradation of piperacillin tazobactam can be minimized and a product having an improved purity can be obtained.

The mixing ratio of piperacillin or a pharmaceutically acceptable salt thereof to tazobactam or a pharmaceutically acceptable salt thereof is approximately 8:1, and may be adjusted as needed. Thus, in the mixture solution prepared in step (1), the ratio of the content of piperacillin sodium or a pharmaceutically acceptable salt thereof calculated as piperacillin free acid to the content of tazobactam or a pharmaceutically acceptable salt thereof calculated as tazobactam free acid is approximately 8:1.

(2) Adding a stabilizer to the solution obtained in the step (1) to give a mixed solution.

It is discovered in the present invention that when preparing piperacillin and tazobactam powder by spray drying method, the stability of piperacillin and tazobactam can be increased by adding a stabilizer to the formulation. The stabilizer used in this invention is selected from one or more selected from the group consisting of carbohydrates, amino acids and polyols. Preferably, polyols are used as the stabilizer, such as polyethylene glycol, glycerol or the like, more preferably, polyethylene glycol, and still further preferably, polyethylene glycol 200-600 and polyethylene glycol 300-400. In this step, based on 100 parts by weight of piperacillin, 0~50 parts by weight of the stabilizer, preferably 0~20 parts by weight of stabilizer, more preferably 2.5~15 parts by weight of stabilizer, and still further preferably 7.5~15 parts by weight of the stabilizer is added.

The concentration calculated as piperacillin of the mixed solution obtained in this step is 1~50% by weight, preferably 1~42% by weight, and more preferably 26~38% by weight.

Aminocarboxylic acid chelating agent EDTA may be further added in this step, if needed. EDTA as a metal chelating agent can increase the stability of piperacillin and tazobactam.

The mixed solution obtained in this step may be used directly for spray drying, or alternatively, may be stored at an appropriate low temperature. The storage temperature is preferably −12~8° C., more preferably −10~0° C. By storing piperacillin and tazobactam solution at an appropriate low temperature, the stability of the solution of piperacillin and tazobactam can be significantly improved, and generation of impurities can be reduced.

The mixed solution obtained in this step may be subjected to sterilization filtration prior to spray drying, if needed, or alternatively, may be used for spray drying directly without the sterilization filtration.

(3) Spray drying the mixed solution obtained in the step (2) to give piperacillin and tazobactam spray-dried powder.

In this step, the spray drying is carried out at in inlet air temperature of 160~240° C., preferably 185~200° C. The spray drying is carried out at an outlet air temperature of 80~130° C., preferably 100~115° C. The heated carrier gas used in the spray drying may be compressed air or nitrogen. Accordingly, piperacillin and tazobactam spray-dried powder is obtained.

The spray-dried powder obtained by the method for industrial production of piperacillin and tazobactam spray-dried powder of the present invention are non-sterile or sterile spherical powder particles, which can be filled into a non-sterile or sterile bag/vial by using a powder filling machine to prepare a non-sterile or sterile API, or a sterile drug product.

The results of an acceleration and long-term stability studies of the spry-dried powder obtained by the method for industrial production of the present invention indicate that the critical quality attributes of the product (such as water, impurities) are comparable to the commercially available reference drug product, and meet the requirements of the Chinese Pharmacopoeia (CP) and/or the United States Pharmacopoeia (USP).

The stabilizer contained in the spray-dried powder obtained by the method for industrialproduction of piperacillin and tazobactam spray-dried powder drying of the present invention has no influence on the biological potency of piperacillin and tazobactam.

The present invention has an advantage that the spray drying process is used for the production of piperacillin and tazobactam powder for the first time. By adding a stabilizer to the formulation, the quality of the spray-dried powder obtained can be comparable to that of the commercially available reference drug product. It is found in the invention that when the storage temperature of a solution of piperacillin and tazobactam is reduced to below 0° C., the generation of impurities can be significantly reduced, and the stability of the solution of piperacillin and tazobactam can be improved. It is confirmed in the present invention that the solution concentration of piperacillin tazobactam is up to 42%. In the present invention, a high concentration solution of piperacillin and tazobactam solution is used for spray drying, and a method for industrial production of piperacillin and tazobactam with high efficiency, high yield and low energy consumption has been developed.

### Examples

The present invention is further described in more details below with reference to the following Examples. However, the present invention is not limited to the Examples in terms of any range of process parameters.

### Example 1: Test for Salt Formation of Piperacillin Acid and Tazobactam Acid by Using Different Bases

### 1.1 Salt Formation by Using Sodium Bicarbonate

42 g of water for injection (WFI) was weighed, and cooled to a temperature of 2~8° C. 0.86 g of citric acid was weighed, and add to WFI. The obtained mixture was stirred until dissolved. Sodium bicarbonate was added so that the pH of the solution reached at about 6.5. 24 g of piperacillin and 3 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. The sample batch number was 200303-2.

### 1.2 Salt Formation by Using Sodium Carbonate

42 g of WFI was weighed, and cooled to 2~8° C. 0.86 g of citric acid was added to WFI. The obtained mixture was stirred until dissolved. Sodium bicarbonate was added so that the pH of the solution reached at about 6.5. 24 g of piperacillin and 3 g of tazobactam were added to the above solution of sodium citrate, and stirred to form a suspension. Sodium carbonate was slowly added so that the pH of the solution was increased to about 6.5. The temperature was maintained at 2~8° C. during the whole process of salt formation. The sample batch number was 200303-3.

The solutions of piperacillin sodium and tazobactam sodium of the above two batches were tested to determine the related substances and content. The results are shown in Table 1.

TABLE 1
Results of salt formation of piperacillin
and tazobactam with different bases

| | Related Substances (%) | | | | |
|---|---|---|---|---|---|
| | Individual | Total | Content (%) | | |
| Batch No. | impurity | impurities | Piperacillin | Tazobactam | Ratio |
| 200303-2 | 0.36 | 0.71 | 35.64 | 4.64 | 8:1 |
| 200303-3 | 0.14 | 0.46 | 36.24 | 4.73 | 8:1 |

According to the result of Table 1, if the salt formation is conducted by using sodium bicarbonate or sodium carbonate, the related substances of the samples meet the CP standard (individual impurity≤2.0%, total impurities ≤4.0%).

### Example 2: Stability Test at Different Solution Concentrations

### 2.1 Stability Test of 42% Solution of Piperacillin and Tazobactam

244 g of WFI was weighed, and cooled to 2~8° C. 6.9 g of citric acid was weighed, and added to WFI. The obtained mixture was stirred until dissolved. Sodium carbonate was added so that the pH of the solution reached at about 6.5. 192 g of piperacillin and 24 g of tazobactam were added to the above solution of sodium citrate, and stirred to form a suspension. Sodium carbonate was slowly added for salt formation, so that the pH of the solution pH was increased to about 6.5. The temperature was maintained at 2~8° C. during the whole process of salt formation. 0.048 g of disodium EDTA and 14.2 g of PEG400 were added. The obtained mixture was stirred until dissolved. Additional water was added until the total weight of the final solution was 500 g. The concentration of the solution concentration was about 42% (calculated as piperacillin). The batch number was 200318-1.

### 2.2 Stability Test of 38% Solution of Piperacillin and Tazobactam

287 g of WFI was weighed, and cooled to 2~8° C. 6.9 g of citric acid was weighed, and added to WFI. The obtained mixture was stirred until dissolved. Sodium carbonate was added so that the pH of the solution reached at about 6.5. 192 g of piperacillin and 24 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0.048 g of disodium EDTA and 14.2 g of PEG 400 were added. The obtained mixture was stirred until dissolved. Additional water was added until the total weight of the final solution was 550 g. The concentration of the solution concentration was about 39% (calculated as piperacillin). The batch number was 200318-2.

### 2.3 Stability Test of 34% Solution of Piperacillin and Tazobactam

338 g of WFI was weighed, and cooled to 2~8° C. 6.9 g of citric acid was weighed, and added to WFI. The obtained mixture was stirred until dissolved. Sodium carbonate was added so that the pH of the solution reached at about 6.5. 192 g of piperacillin and 24 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0.048 g of disodium EDTA and 14.2 g of PEG4 00 were added. The obtained mixture was stirred until dissolved. Additional water was added until the total weight of the final solution was 600 g. The concentration of the solution was about 34% (calculated as piperacillin). The batch number was 200318-3.

### 2.4 Stability Test of 30% Solution of Piperacillin and Tazobactam

317 g of WFI was weighed, and cooled to 2~8° C. 5.5 g of citric acid was weighed, and added to WFI. The obtained mixture was stirred until dissolved. Sodium carbonate was added so that the pH of the solution reached at about 6.5. 152 g of piperacillin and 19 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0.038 g of disodium EDTA and 11.4 g of PEG 400 were added. The obtained mixture was stirred until dissolved. Additional water was added until the total weight of the final solution was 524 g. The concentration of the solution was about 30% (calculated as piperacillin). The batch number was 200319-1.

### 2.5 Stability Test of 26% Solution of Piperacillin and Tazobactam

358 g of WFI was weighed, and cooled to 2~8° C. 5.2 g of citric acid was weighed, and added to WFI. The obtained mixture was stirred until dissolved. Sodium carbonate was added so that the pH of the solution reached at about 6.5. 144 g of piperacillin and 18 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0.036 g of disodium EDTA and 10.8 g of PEG 400 were added. The obtained mixture was stirred until dissolved. Additional water was added until the total weight of the final solution was 554 g. The concentration of the solution was about 30% (calculated as piperacillin). The batch number was 200319-2.

The above 5 batches of the solution of piperacillin and tazobactam were filtered using a 0.22 μm microporous filter membrane. The filtration time was recorded to calculate the filtration efficiency. The filtered solutions were studied for the stability at 2-8° C. The solutions were sampled to determine the pH, content and related substances at 0 h, 24 h, 48 h and 72 h, respectively. The results are shown in Table 2.

TABLE 2
Test result of the solution stability

| | Con- | | | | Related substances | | | | |
|---|---|---|---|---|---|---|---|---|---|
| | cen- | Filtration | | | (%) | | | | |
| Batch | tration | efficiency | | | Individual | Total | Content (%) | | |
| No. | (%) | (g/min) | Time | pH | impurity | impurities | Pip | Taz | Ratio |
| 200318-1 | 42 | 0.83 | 0 h | 7.7 | 0.14 | 0.48 | 43.15 | 5.56 | 8:1 |
| | | | 24 h | 7.5 | 0.27 | 0.65 | 43.04 | 5.58 | 8:1 |
| | | | 48 h | 7.4 | 0.44 | 0.87 | 41.54 | 5.42 | 8:1 |
| | | | 72 h | 7.2 | 0.56 | 1.05 | 41.90 | 5.41 | 8:1 |
| 200318-2 | 38 | 0.83 | 0 h | 6.7 | 0.15 | 0.48 | 37.50 | 4.86 | 8:1 |
| | | | 24 h | 6.6 | 0.22 | 0.67 | 37.79 | 4.94 | 8:1 |
| | | | 48 h | 6.8 | 0.32 | 0.88 | 37.60 | 4.93 | 8:1 |
| | | | 72 h | 6.7 | 0.42 | 1.13 | 37.18 | 4.83 | 8:1 |
| 200318-3 | 34 | 6.4 | 0 h | 7.3 | 0.15 | 0.45 | 33.96 | 4.38 | 8:1 |
| | | | 24 h | 7.1 | 0.21 | 0.61 | 34.38 | 4.46 | 8:1 |
| | | | 48 h | 7.2 | 0.32 | 0.76 | 34.28 | 4.46 | 8:1 |
| | | | 72 h | 7.1 | 0.40 | 0.89 | 33.93 | 4.37 | 8:1 |
| 200319-1 | 30 | 9.0 | 0 h | 7.1 | 0.16 | 0.49 | 30.30 | 3.98 | 8:1 |
| | | | 24 h | 7.2 | 0.24 | 0.63 | 30.16 | 3.98 | 8:1 |
| | | | 48 h | 7.1 | 0.32 | 0.76 | 29.89 | 3.91 | 8:1 |
| | | | 72 h | 7.0 | 0.73 | 1.49 | 29.90 | 3.96 | 8:1 |
| 200319-2 | 26 | 8.5 | 0 h | 6.9 | 0.16 | 0.46 | 27.20 | 3.53 | 8:1 |
| | | | 24 h | 6.6 | 0.17 | 0.57 | 27.08 | 3.52 | 8:1 |
| | | | 48 h | 6.7 | 0.22 | 0.67 | 27.12 | 3.50 | 8:1 |
| | | | 72 h | 6.7 | 0.49 | 1.31 | 26.94 | 3.51 | 8:1 |

As can be seen from Table 2:
1) As the solution concentration decreases, the filtration efficiency is increased. The filtration efficiency of the solution with a concentration below 38% is significantly higher than that with concentration above 38%.
2) In the solution of each of the 5 groups having different concentrations (26%~42%, calculated as piperacillin) stored at 2~8° C. for 72 h, the related substances can meet the CP standards. Among them, group 3 (with a concentration of about 34%) has the smallest impurity increment.

### Example 3: Spray Drying Test of Piperacillin and Tazobactam with Different Stabilizers (with a Solution Concentration of 35%)

3.1 Spray Drying Test of Piperacillin and Tazobactam without a Stabilizer

225 g of WFI was weighed, and cooled to 2~8° C. 4.6 g of citric acid was weighed, and added to WFI. The obtained mixture was stirred until dissolved. Sodium carbonate was added so that the pH of the solution reached at about 6.5. 128 g of piperacillin and 16 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0,032 g of disodium EDTA was added. The obtained mixture was stirred until dissolved. Additional water was added until the total weight of the final solution was 400 g. The solution was filtered through a 0.22 μm filter and held for future use. The batch number was 200424-1.

### 3.2 Spray Drying Test of Piperacillin and Tazobactam Containing PEG400 as Stabilizer

225 g of WFI was weighed, and cooled to 2~8° C. 4.6 g of citric acid was weighed, and added to WFI. The obtained mixture was stirred until dissolved. Sodium carbonate was added so that the pH of the solution reached at about 6.5. 128 g of piperacillin and 16 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0.032 g of disodium EDTA and 9.6 g of PEG 400 were added. The obtained mixture was stirred until dissolved. Additional water was added until the total weight of the final solution was 400 g. The solution was filtered through a 0.22 μm filter and held for future use. The solution contained 7.5% of PEG 400 (based on piperacillin). The batch number was 200424-3.

### 3.3 Spray Drying Test of Piperacillin and Tazobactam Containing PEG 300 as a Stabilizer

162 g of WFI was weighed, and cooled to 2~8° C. 3.45 g of citric acid was weighed, and added to WFI. The obtained mixture was stirred until dissolved. Sodium carbonate was added so that the pH of the solution reached at about 6.5. 96 g of piperacillin and 12 g of tazobactam were added to the above solution of sodium citrate, and stirred to form a suspension. Sodium carbonate was slowly added for salt formation so that the pH of the solution was increased to about 6.5. The temperature was maintained at 2~8° C. during the whole process of salt formation. 0.024 g of disodium EDTA and 7.2 g of PEG 300 were added. The obtained mixture was stirred until dissolved. Additional water was added until the total weight of the final solution was 300 g. The solution was filtered through a 0.22 μm filter and held for future use. The solution contained 7.5% of PEG 300. The batch number was 200425-4.

### 3.4 Spray Drying Test of Piperacillin and Tazobactam Containing PEG600 as Stabilizer

162 g of WFI was weighed, and cooled to 2~8° C. Around 3. to 6.5 of citric acid was weighed. The temperature was maintained at 45 g during the whole process of salt formation. The citric acid was added to WFI. The obtained mixture was stirred until dissolved. Sodium carbonate was added so that the pH of the solution reached at about 6.5. 96 g of piperacillin and 12 g of tazobactam were added to the above solution of sodium citrate, and stirred to form a suspension. Sodium carbonate was slowly added for salt formation so that the pH of the solution was increased at 2~8° C. 0.024 g of disodium EDTA and 7.2 g of PEG 600 were added. The obtained mixture was stirred until dissolved. Additional water was added until the total weight of the final solution was 300 g. The solution was filtered through a 0.22 μm filter and held for future use. The solution contained 7.5% of PEG 600, The batch number was 200425-5.

The solution of the above 4 batches were spray dried by using a Lab scale spray drying equipment with the parameters set as follows. Inlet air temperature: 185° C.; Outlet air temperature: 115° C. The obtained spray-dried powder was placed at 80° C. for a super-accelerated test. The spray-dried powders were sampled to determine the related substances, water and pH at Day 0, Day 1 and Day 3, The test results are shown in Table 3.

TABLE 3
Stability result of piperacillin and tazobactam
spray-dried powders with different stabilizers

| | | Batch No. | | | |
|---|---|---|---|---|---|
| | Excipient | 200424-1 | 200424-3 | 200425-4 | 200425-5 |
| | and | no | 7.5% | 7.5% | 7.5% |
| Formulation | proportion | stabilizer | PEG 400 | PEG 300 | PEG 600 |
| Water (%) | Day 0 | 1.55 | 1.24 | 1.03 | 1.35 |
| pH | Day 0 | 6.42 | 6.26 | 6.25 | 7.36 |
| Individual | Day 0 | 0.16 | 0.15 | 0.13 | 0.28 |
| impurity | Day 1 | 0.69 | 0.43 | 0.41 | 0.66 |
| (%) | Day 3 | 1.46 | 0.99 | 0.87 | 1.14 |
| | Variation | 1.30 | 0.85 | 0.75 | 0.86 |
| | in 3 days | | | | |
| Total | Day 0 | 0.71 | 0.64 | 0.64 | 0.97 |
| impurities | Day 1 | 2.15 | 1.77 | 1.57 | 1.91 |
| (%) | Day 3 | 4.98 | 3.66 | 3.36 | 3.50 |
| | Variation | 4.28 | 3.02 | 2.73 | 2.54 |
| | in 3 days | | | | |

As can be seen from Table 3, as compared with the batch of piperacillin and tazobactam without PEG, the spray-dried powder obtained by spray drying a solution with PEG 400, PEG 300, PEG 600 added has a lower water content, a lower individual impurity level and a lower level of total impurities, indicating that the spray-dried powder with PEG added has a lower water content and an enhanced stability.

### Example 4: Spray Drying Test of Piperacillin and Tazobactam with Different Concentrations of PEG 400

By using citric acid and sodium bicarbonate for salt formation/pH adjustment, the effect of different proportions of PEG 400 on product quality was examined to determine an optimal proportion of PEG 400. A total of 9 experiments were conducted.

4.1 Spray Drying Test of Piperacillin and Tazobactam without PEG 400

225 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA was added. The obtained mixture was stirred until dissolved. Additional water was added until the total weight of the final solution was 400 g. The solution was filtered through a 0.22 μm filter and held for future use. The solution contained no PEG 400. The batch number was 200521-1.

4.2 Spray Drying Test of Piperacillin and Tazobactam with 2.5% PEG 400

225 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 3.2 g of PEG 400 were added. The obtained mixture was stirred until dissolved. Additional water was added until the total weight of the final solution was 400 g. The solution was filtered through a 0.22 μm filter and held for future use. The solution contained 2.5% of PEG 400. The batch number was 200521-2.

4.3 Spray Drying Test of Piperacillin and Tazobactam with 5% PEG 400

225 g of WFI was weighed, and cooled to 2-8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 6.4 g of PEG 400 were added. The obtained mixture was stirred until dissolved. Additional water was added until the total weight of the final solution was 400 g. The solution was filtered through a 0.22 μm filter and held for future use. The solution contained 5% of PEG 400. The batch number was 200521-3.

4.4 Spray Drying Test of Piperacillin and Tazobactam with 7.5% PEG 400

225 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 9.6 g of PEG 400 were added. The obtained mixture was stirred until dissolved. Additional water was added until the total weight of the final solution was 400 g. The solution was filtered through a 0.22 μm filter and held for future use. The solution contained 7.5% of PEG 400. The batch number was 200522-1.

4.5 Spray Drying Test of Piperacillin and Tazobactam with 10% PEG 400

222 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 12.8 g of PEG 400 were added. The obtained mixture was stirred until dissolved. Additional water was added until the total weight of the final solution was 400 g. The solution was filtered through a 0.22 μm filter and held for future use. The solution contained 10% of PEG 400. The batch number was 200522-2.

4.6 Spray Drying Test of Piperacillin and Tazobactam with 12.5% PEG 400

219 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 16 g of PEG 400 were added. The obtained mixture was stirred until dissolved. Additional water was added until the total weight of the final solution was 400 g. The solution was filtered through a 0.22 μm filter and held for future use. The solution contained 12.5% of PEG 400. The batch number was 200523-1.

4.7 Spray Drying Test of Piperacillin and Tazobactam with 15% PEG 400

216 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 19.2 g of PEG 400 were added. The obtained mixture was stirred until dissolved. Additional water was added until the total weight of the final solution was 400 g. The solution was filtered through a 0.22 μm filter and held for future use. The solution contained 15% of PEG 400. The batch number was 200523-2.

4.8 Spray Drying Test of Piperacillin and Tazobactam with 17.5% PEG 400

213 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 22.4 g of PEG 400 were added. The obtained mixture was stirred until dissolved. Additional water was added until the total weight of the final solution was 400 g. The solution was filtered through a 0.22 μm filter and held for future use. The solution contained 17.5% of PEG 400. The batch number was 200523-3.

4.9 Spray Drying Test of Piperacillin and Tazobactam with 20% PEG 400

210 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 25.6 g of PEG 400 were added. The obtained mixture was stirred until dissolved. Additional water was added until the total weight of the final solution was 400 g. The solution was filtered through a 0.22 μm filter and held for future use. The solution contained 20% of PEG 400. The batch number was 200523-4.

The solution of the above 9 batches were spray dried by using a Lab scale spray drying equipment with the parameters set as follows. Inlet air temperature: 185° C.; Outlet air temperature: 115° C. The obtained spray-dried powder was placed at 80° C. for a super-accelerated test. The spray-dried powders were sampled to determine the water, related substances, pH and reconstitution time at Day 0, Day 1 and Day 3. The test results are shown in Table 4.

TABLE 4
Stability result of piperacillin and tazobactam spray-dried powders with different concentrations of PEG 400

| | | Group | | | | | | | | |
|---|---|---|---|---|---|---|---|---|---|---|
| | | 1 | 2 | 3 | 4 | 5 | 6 | 7 | 8 | 9 |
| Batch No. | | 200521-1 | 200521-2 | 200521-3 | 200522-1 | 200522-2 | 200523-1 | 200523-2 | 200523-3 | 200523-4 |
| Formulation | PEG 400 | 0% | 2.5% | 5% | 7.5% | 10% | 12.5% | 15% | 17.5% | 20% |
| | proportion | PEG400 | PEG400 | PEG400 | PEG400 | PEG400 | PEG400 | PEG400 | PEG400 | PEG400 |
| Water (%) | Day 0 | 1.81 | 1.84 | 1.83 | 1.25 | 1.25 | 1.10 | 1.11 | 1.04 | 1.04 |
| pH | Day 0 | 6.32 | 6.39 | 7.43 | 6.56 | 6.51 | 6.89 | 6.60 | 6.88 | 6.50 |
| Individual | Day 0 | 0.17 | 0.17 | 0.24 | 0.14 | 0.14 | 0.14 | 0.14 | 0.20 | 0.16 |
| impurity | Day 1 | 0.74 | 0.64 | 0.71 | 0.48 | 0.45 | 0.62 | 0.60 | 0.81 | 0.80 |
| (%) | Day 3 | 1.16 | 0.99 | 1.08 | 0.74 | 0.76 | 0.87 | 0.92 | 1.15 | 1.14 |
| | Variation | 1.00 | 0.82 | 0.84 | 0.60 | 0.62 | 0.73 | 0.78 | 0.95 | 0.98 |
| | in 3 days | | | | | | | | | |
| Total | Day 0 | 0.75 | 0.73 | 0.99 | 0.67 | 0.64 | 0.71 | 0.70 | 0.85 | 0.84 |
| impurities | Day 1 | 2.30 | 2.13 | 2.17 | 1.83 | 1.70 | 1.98 | 2.09 | 2.75 | 3.22 |
| (%) | Day 3 | 3.64 | 3.20 | 3.53 | 2.53 | 2.78 | 2.81 | 3.45 | 4.07 | 5.18 |
| | Variation | 2.90 | 2.47 | 2.54 | 1.86 | 2.14 | 2.11 | 2.75 | 3.22 | 4.35 |
| | in 3 days | | | | | | | | | |

| | | | | | | | | | |
|---|---|---|---|---|---|---|---|---|---|
| Reconstitution time | 3 | 2 | 3 | 3 | 3 | 2 | 3 | 2 | 2 |
| (min) (4.5 g, dissolved | | | | | | | | | |
| in 50 ml WFI) | | | | | | | | | |

It can be seen from Table 4:
(1) The addition of PEG 400 in an amount of 0%~20% can effectively reduce the water content of the spray-dried powder. The higher the PEG 400 ratio is, the lower the water content is. The water meets the requirements of the Chinese Pharmacopoeia (water≤2.5%).
(2) With the increase of PEG 400 proportion from 0% to 20%, after a storage at 80° C. for 3 days, the increment of individual impurity and total impurities presented a trend of first decreasing and then increasing. When PEG400 accounted for 2.5~15%, the 3-day increment of impurity (individual impurity and total impurities) is less than that of the sample without PEG 400, and meet the requirements of the Chinese Pharmacopoeia (individual impurity≤2.0%, and total impurities≤4.0%).
(3) The reconstitution time of the spray-dried powder with PEG 400 is about 2~3 min.

### Example 5: Stability Test of Solutions of Piperacillin and Tazobactam at Different Storage Temperatures

### 5.1 Stability Study of a Solution of Piperacillin and Tazobactam at the Storage Temperature of 2~8° C.

225 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 10.24 g of PEG 400 were added. The obtained mixture was stirred until dissolved. Additional water was added until the total weight of the final solution was 400 g. The solution batch number was 200617-2. The solution of piperacillin and tazobactam was placed in a refrigerator at 2~8° C. for a stability study of the solution. The solution was sampled at 0 h, 24 h, 48 h and 72 h, respectively, to determine the related substances. The results are shown in Table 5.

### 5.2 Stability Study of a Solution of Piperacillin and Tazobactam Solution at the Storage Temperature of −5° C.

225 g of WFI was weighed, and cooled to 2~8° C. 6.4 g of citric acid was weighed, and added to WFI. The obtained mixture was stirred until dissolved. Sodium bicarbonate was added so that the pH of the solution reached at about 6.5. 128 g of piperacillin and 16 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.032 g of disodium EDTA and 10.24 g of PEG 400 were added. The obtained mixture was stirred until dissolved. Additional water was added until the total weight of the final solution was 400 g. The solution batch number was 200729. The solution of piperacillin and tazobactam was placed at −5° C. for a stability study of the solution. The solution was sampled at 0 h, 24 h, 48 h and 72 h, respectively, to determine the related substances. The results are shown in Table 5.

TABLE 5
Stability result of solution of piperacillin and
tazobactam at different storage temperatures

| Time | | 0 h | 24 h | 48 h | 72 h |
|---|---|---|---|---|---|
| 200617-2 | Individual impurity (%) | 0.13 | 0.23 | 0.32 | 0.42 |
| (stored at | Absolute increment of | / | 0.10 | 0.19 | 0.29 |
| 2~8° C.) | individual impurity over 0 h (%) | | | | |
| | Total impurities (%) | 0.62 | 0.72 | 0.91 | 1.11 |
| | Absolute increment of total | / | 0.10 | 0.29 | 0.49 |
| | impurities over 0 h (%) | | | | |
| 200729 | Individual impurity (%) | 0.12 | 0.16 | 0.20 | 0.24 |
| (stored | Absolute increment of | / | 0.04 | 0.08 | 0.12 |
| at −5° C.) | individual impurity over 0 h (%) | | | | |
| | Total impurities (%) | 0.68 | 0.87 | 0.92 | 0.97 |
| | Absolute increment of total | / | 0.19 | 0.24 | 0.28 |
| | impurities over 0 h (%) | | | | |

It can be seen from Table 5 that, as compared with the storage condition at 2~8° C., the trend of impurity increase slows down at the storage condition at −5° C., and the impurity increment is reduced by about half within 72 h. Therefore, in a commercial production, reducing the storage temperature (such as, at −12~0° C.) in preparation, sterilization filtration, solution storage may be considered to reduce the generation of degradation impurities.

### Example 6: Process Parameter (Outlet Air Temperature) Study in the Spray Drying of Piperacillin and Tazobactam

450 g of WFI was weighed, and cooled to 2~8° C. 12.8 g of citric acid was weighed, and added to WFI. The obtained mixture was stirred until dissolved. Sodium bicarbonate was added so that the pH of the solution reached at about 6.5. 256 g of piperacillin and 32 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.064 g of disodium EDTA and 20.48 g of PEG 400 were added. The obtained mixture was stirred until dissolved. Additional water was added until the total weight of the final solution was 800 g.

### 6.1 Spray Drying Test at an Inlet Air Temperature of 185° C. And an Outlet Air Temperature of 115° C.

200 ml of the above solution of piperacillin and tazobactam solution was spray dried by using a Lab scale spray drying equipment with the parameters set as follows. Inlet air temperature: 185° C.; Outlet air temperature: 115° C. The batch number of the obtained spray-dried powder was 200710-1.

### 6.2 Spray Drying Test at an Inlet Air Temperature of 185° C. And an Outlet Air Temperature of 110° C.

200 ml of the above solution of piperacillin and tazobactam solution was spray dried by using a Lab scale spray drying equipment with the parameters set as follows. Inlet air temperature: 185° C.; Outlet air temperature: 110° C. The batch number of the obtained spray-dried powder was 200710-2.

### 6.3 Spray Drying Test at an Inlet Air Temperature of 185° C. And an Outlet Air Temperature of 105° C.

200 ml of the above solution of piperacillin and tazobactam solution was spray dried by using a Lab scale spray drying equipment with the parameters set as follows. Inlet air temperature: 185° C.; Outlet air temperature: 105° C. The batch number of the obtained spray-dried powder was 200710-3.

### 6.4 Spray Drying Test at an Inlet Air Temperature of 185° C. And an Outlet Air Temperature of 100° C.

200 ml of the above solution of piperacillin and tazobactam solution was spray dried by using a Lab scale spray drying equipment with the parameters set as follows. Inlet air temperature: 185° C.; Outlet air temperature: 100° C. The batch number of the obtained spray-dried powder was 200710-4.

The above 4 batches of piperacillin tazobactam spray-dried powder were tested to determine the water and related substances. The results are shown in Table 6.

TABLE 6
Quality study result of piperacillin and tazobactam spray-dried powders
obtained by spray dyring at different outlet air temperatures

| | | Batch No. | | | |
|---|---|---|---|---|---|
| | | 200710-1 | 200710-2 | 200710-3 | 200710-4 |
| | | Inlet: | Inlet: | Inlet: | Inlet: |
| | | 185° C. | 185° C. | 185° C. | 185° C. |
| | | Outlet: | Outlet: | Outlet: | Outlet: |
| | | 115° C. | 110° C. | 105° C. | 100° C. |
| Water (%) | | 1.6 | 1.9 | 2.2 | 2.3 |
| Related | Individual | 0.14 | 0.15 | 0.26 | 0.15 |
| substances | impurity | | | | |
| | (%) | | | | |
| | Total | 0.90 | 0.88 | 1.19 | 0.81 |
| | impurities | | | | |
| | (%) | | | | |

It can be seen from Table 6 that when the inlet air temperature is 185° C. and the outlet air temperature is 100-115° C., the quality of the spray-dried powders meet the requirements of the Chinese Pharmacopoeia (water≤2.5%, individual impurity≤2.0%, total impurities≤4.0%).

### Example 7: Process Parameter (Iutlet Air Temperature) Study in the Spray Drying of Piperacillin and Tazobactam

450 g of WFI was weighed, and cooled to 2-8° C. 12.8 g of citric acid was weighed, and added to WFI. The obtained mixture was stirred until dissolved. Sodium bicarbonate was added so that the pH of the solution reached at about 6.5. 256 g of piperacillin and 32 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.064 g of disodium EDTA and 20.48 g of PEG 400 were added. The obtained mixture was stirred until dissolved. Additional water was added until the total weight of the final solution was 800 g.

### 7.1 Spray Drying Test at an Inlet Air Temperature of 185° C. And an Outlet Air Temperature of 115° C.

200 ml of the above solution of piperacillin and tazobactam solution was spray dried by using a Lab scale spray drying equipment with the parameters set as follows. Inlet air temperature: 185° C.; Outlet air temperature: 115° C. The batch number of the obtained spray-dried powder was 200713-1.

### 7.2 Spray Drying Test at an Inlet Air Temperature of 190° C. And an Outlet Air Temperature of 115° C.

200 ml of the above solution of piperacillin and tazobactam solution was spray dried by using a Lab scale spray drying equipment with the parameters set as follows. Inlet air temperature: 190° C.; Outlet air temperature: 115° C. The batch number of the obtained spray-dried powder was 200713-2.

### 7.3 Spray Drying Test at an Inlet Air Temperature of 195° C. And an Outlet Air Temperature of 115° C.

200 ml of the above solution of piperacillin and tazobactam solution was spray dried by using a Lab scale spray drying equipment with the parameters set as follows. Inlet air temperature: 195° C.; Outlet air temperature: 115° C. The batch number of the obtained spray-dried powder was 200713-3.

### 7.4 Spray Drying Test at an Inlet Air Temperature of 200° C. And an Outlet Air Temperature of 115° C.

200 ml of the above solution of piperacillin and tazobactam solution was spray dried by using a Lab scale spray drying equipment with the parameters set as follows. Inlet air temperature: 200° C.; Outlet air temperature: 115° C. The batch number of the obtained spray-dried powder was 200713-4.

The above 4 batches of piperacillin tazobactam spray-dried powder were tested to determine the water and related substances. The results are shown in Table 7.

TABLE 7
Quality study result of piperacillin and tazobactam spray-dried powders
obtained by spray dyring at different inlet air temperatures

| | | Batch No. | | | |
|---|---|---|---|---|---|
| | | 200713-1 | 200713-2 | 200713-3 | 200713-4 |
| | | Inlet: | Inlet: | Inlet: | Inlet: |
| | | 185° C. | 190° C. | 195° C. | 200° C. |
| | | Outlet: | Outlet: | Outlet: | Outlet: |
| | | 115° C. | 115° C. | 115° C. | 115° C. |
| Water (%) | | 1.5 | 1.4 | 1.5 | 1.5 |
| Related | Individual | 0.13 | 0.14 | 0.15 | 0.15 |
| substances | impurity | | | | |
| | (%) | | | | |
| | Total | 0.75 | 0.79 | 0.83 | 0.84 |
| | impurities | | | | |
| | (%) | | | | |

It can be seen from Table 7 that when the outlet air temperature is 115° C. and the inlet air temperature is 185-200° C., there is no significant difference in the quality between the spray-dried powders, and the spray-dried powders meet the requirements of the Chinese Pharmacopoeia (water ≤2.5%, individual impurity <2.0%, total impurities ≤4.0%).

### Example 8: Scaling Up and Stability Study in the Spray Drying of Piperacillin and Tazobactam

Solutions of piperacillin and tazobactam without a stabilizer and with PEG 400 were prepared. The spray drying was scaled up by using a pilot scale spray drying equipment. An accelerated and long-term stability study was performed by testing according to the method of the USP for a comparison with a commercially available reference drug product (RLD, Wyeth Zosyn).

8.1 Scaling Up and Stability Study in the Spray Drying of Piperacillin and Tazobactam without PEG400

1410 g of WFI was weighed, and cooled to 2~8° C., 40 g of citric acid was weighed, and added to WFI. The obtained mixture was stirred until dissolved. Sodium bicarbonate was added so that the pH of the solution reached at about 6.5. 800 g of piperacillin and 100 g of tazobactam were added to the above solution of sodium citrate, and stirred to form a suspension. Sodium bicarbonate was slowly added for salt formation so that the pH of the solution was increased to about 6.5. The temperature was maintained at 2~8° C. during the whole process of salt formation. 0.2 g of disodium EDTA was added. The obtained mixture was stirred until dissolved. Additional water was added until the total weight of the final solution was 2600 g. The solution was filtered through a 0.22 μm filter and held for future use. The batch number was 200808-1.

8.2 Scaling Up and Stability Study in the Spray Drying of Piperacillin and Tazobactam with PEG 400

1410 g of WFI was weighed, and cooled to 2~8° C. 40 g of citric acid was weighed, and added to WFI. The obtained mixture was stirred until dissolved. Sodium bicarbonate was added so that the pH of the solution reached at about 6.5. 800 g of piperacillin and 100 g of tazobactam were added to the above solution of sodium citrate, and stirred to form a suspension, Sodium bicarbonate was slowly added for salt formation so that the pH of the solution was increased to about 6.5. The temperature was maintained at 2~8° C. during the whole process of salt formation. 0.2 g of disodium EDTA and 64 g of PEG 400 were added. The obtained mixture was stirred until dissolved. Additional water was added until the total weight of the final solution was 2600 g. The solution was filtered through a 0.22 μm filter and held for future use. The batch number was 200808-2.

The solutions of the above two batches were spray dried by using a pilot scale spray drying equipment with the parameters set as follows. Inlet air temperature: 195° C.; Outlet air temperature: 110° C. For the stability study, the obtained spray-dried powders were packaged into drug products, and placed at an accelerated test condition (40±2° C./75±5% RH) and at a long-term test condition (25° C./60% RH), respectively. The sampling time points in the accelerated test were 0, 2, 3 and 6 months. The sampling time points in the long-term test were 0, 3, 6 and 9 months. The samples were tested to determine the pH, water, related substances and content according to the method of the USP. The results are shown in Table 8.

TABLE 8
Result of accelerated stability study of piperacillin and tazobactam spray-dried powder at 40 ± 2° C./75 ± 5% RH

| | | | Batch No. | | | | | | | | | | | |
|---|---|---|---|---|---|---|---|---|---|---|---|---|---|---|
| | | | 200808-1 | | | | 200808-2 | | | | ALM9/11 (RLD) | | | |
| Accelerated stability test | | | Stabilizer content in the formulation | | | | | | | | | | | |
| | | Accept- | Spray-dried powder | | | | Spray-dried powder | | | | Lyophilized powder | | | |
| | | ance | without PEG400 | | | | with PEG400 | | | | without a stabilizer | | | |
| Test items | | criteria | 0 M | 2 M | 3 M | 6 M | 0 M | 2 M | 3 M | 6 M | 0 M | 2 M | 3 M | 6 M |
| pH | | 5.0-7.0 | 6.8 | 6.3 | 6.2 | 6.0 | 6.7 | 6.7 | 6.5 | 6.3 | 6.3 | 6.3 | 6.2 | 6.1 |
| Water (%) | | ≤2.5 | 2.4 | 2.4 | 2.4 | 2.8 | 1.6 | 1.8 | 1.6 | 1.6 | 0.8 | 0.8 | 0.7 | 0.7 |
| Reconstitution | | / | 3 | 3 | 3 | 2 | 2 | 3 | 2 | 2 | 2 | 4 | 2 | 2 |
| time (min) | | | | | | | | | | | | | | |
| Im- | Tazobactam | ≤0.3 | 0.08 | 0.23 | 0.55 | 0.44 | 0.05 | 0.10 | 0.15 | 0.18 | 0.09 | 0.12 | 0.15 | 0.19 |
| purities | related | | | | | | | | | | | | | |
| (%) | substance A | | | | | | | | | | | | | |
| | Amoxicillin | ≤0.2 | N.D. | N.D. | N.D. | N.D. | N.D. | N.D. | N.D. | N.D. | N.D. | N.D. | N.D. | N.D. |
| | related | | | | | | | | | | | | | |
| | substance A | | | | | | | | | | | | | |
| | Piperacillin | ≤0.8 | 0.18 | 0.29 | 0.37 | 0.42 | 0.11 | 0.18 | 0.20 | 0.25 | 0.14 | 0.27 | 0.32 | 0.41 |
| | related | | | | | | | | | | | | | |
| | substance E | | | | | | | | | | | | | |
| | Formyl | ≤0.2 | N.D. | N.D. | N.D. | N.D. | N.D | N.D. | N.D. | N.D. | N.D. | N.D. | N.D | N.D |
| | penicill- | | | | | | | | | | | | | |
| | amine | | | | | | | | | | | | | |
| | Ampicillin | ≤0.2 | 0.04 | 0.06 | 0.07 | 0.08 | N.D. | 0.03 | 0.03 | 0.03 | 0.04 | 0.06 | 0.08 | 0.10 |
| | Piperazine- | ≤0.2 | N.D. | N.D. | N.D. | N.D. | N.D. | N.D. | N.D | N.D. | N.D. | N.D. | N.D. | N.D. |
| | dione- | | | | | | | | | | | | | |
| | carbonyl | | | | | | | | | | | | | |
| | D-phenyl- | | | | | | | | | | | | | |
| | glycyl- | | | | | | | | | | | | | |
| | penicillin | | | | | | | | | | | | | |
| | Acetylated | ≤0.5 | 0.03 | 0.09 | 0.12 | 0.07 | 0.03 | 0.05 | 0.05 | 0.04 | 0.04 | 0.07 | 0.08 | 0.06 |
| | piperacillin | | | | | | | | | | | | | |
| | penicilloic | | | | | | | | | | | | | |
| | acid | | | | | | | | | | | | | |
| | Piperacillin | ≤0.15 | 0.08 | N.D. | 0.13 | 0.50 | 0.07 | N.D. | N.D | 0.12 | 0.10 | N.D. | 0.03 | 0.13 |
| | penicilloic | | | | | | | | | | | | | |
| | acid | | | | | | | | | | | | | |
| | isomer 1 | | | | | | | | | | | | | |
| | Piperacillin | ≤1.5 | 0.48 | 1.40 | 2.46 | 2.33 | 0.32 | 0.56 | 0.72 | 0.80 | 0.45 | 0.65 | 0.82 | 0.99 |
| | penicilloic | | | | | | | | | | | | | |
| | acid | | | | | | | | | | | | | |
| | isomer 2 | | | | | | | | | | | | | |
| | L-Piper- | — | N.D. | N.D. | 0.08 | N.D. | N.D. | N.D. | N.D | N.D. | N.D. | N.D. | 0.06 | N.D. |
| | acillin | | | | | | | | | | | | | |
| | Piperacillin | ≤0.5 | 0.05 | 0.39 | 0.95 | 1.05 | 0.04 | 0.15 | 0.22 | 0.29 | 0.03 | 0.05 | 0.07 | 0.12 |
| | penilloic | | | | | | | | | | | | | |
| | acid | | | | | | | | | | | | | |
| | Piperacillin | — | 0.08 | 0.15 | 0.09 | 0.09 | 0.08 | 0.10 | 0.08 | 0.09 | N.D. | N.D. | N.D. | N.D. |
| | methyl ester | | | | | | | | | | | | | |
| | Piperacillin | ≤0.2 | 0.03 | 0.03 | 0.03 | 0.04 | 0.03 | 0.03 | 0.05 | 0.04 | 0.04 | 0.04 | 0.04 | 0.04 |
| | dimer | | | | | | | | | | | | | |
| | ethyl ester | | | | | | | | | | | | | |
| | Piperacillin | ≤0.2 | N.D. | N.D. | N.D. | N.D. | N.D. | N.D. | N.D. | N.D. | N.D. | N.D. | N.D. | N.D. |
| | dimer | | | | | | | | | | | | | |
| | thiazolamide | | | | | | | | | | | | | |
| | derivative | | | | | | | | | | | | | |
| | Piperacillin | ≤0.3 | 0.02 | N.D. | 0.02 | N.D. | 0.02 | 0.02 | 0.02 | 0.02 | N.D. | N.D. | N.D. | N.D. |
| | penicill- | | | | | | | | | | | | | |
| | amide | | | | | | | | | | | | | |
| | Piperacillin | ≤0.4 | 0.04 | 0.10 | 0.19 | 0.13 | 0.04 | 0.11 | 0.10 | 0.10 | 0.10 | 0.11 | 0.17 | 0.14 |
| | dimer | | | | | | | | | | | | | |
| | Piper- | ≤0.3 | 0.33 | 0.33 | 0.30 | 0.31 | 0.33 | 0.35 | 0.31 | 0.31 | 0.22 | 0.23 | 0.22 | 0.22 |
| | acillinyl- | | | | | | | | | | | | | |
| | ampicillin | | | | | | | | | | | | | |
| | Individual | ≤0.1 | 0.06 | 0.09 | 0.23 | 0.16 | 007 | 0.07 | 0.08 | 0.06 | 0.06 | 0.08 | 0.09 | 0.07 |
| | impurity | | | | | | | | | | | | | |
| | Total | ≤4.0 | 1.71 | 3.62 | 7.14 | 6.63 | 1.36 | 1.93 | 2.61 | 2.90 | 1.45 | 1.78 | 2.53 | 2.81 |
| | impurities | | | | | | | | | | | | | |
| Cotent | Piperacillin | 90.0~ | 103.5 | 97.8 | 94.4 | 95.0 | 103.3 | 99.8 | 99.8 | 99.2 | 102.3 | 98.0 | 99.0 | 98.7 |
| (%) | | 110.0 | | | | | | | | | | | | |
| | Tazobactam | 90.0~ | 103.0 | 100.6 | 90.3 | 92.4 | 102.8 | 101.8 | 102.0 | 100.9 | 101.1 | 99.0 | 99.9 | 96.8 |
| | | 110.0 | | | | | | | | | | | | |
| | Piperacillin: | 8:1 | 8:1 | 8:1 | 8:1 | 8:1 | 8:1 | 8:1 | 8:1 | 8:1 | 8:1 | 8:1 | 8:1 | 8:1 |
| | Tazobactam | | | | | | | | | | | | | |

TABLE 9
Result of long-term stability study of piperacillin and tazobactam spray-dried powder at 25 ± 2° C./60 ± 5% RH

| | | | Batch No. | | | | | | | | | | | |
|---|---|---|---|---|---|---|---|---|---|---|---|---|---|---|
| | | | 200808-1 | | | | 200808-2 | | | | ALM9/11 (RLD) | | | |
| Long-term stability test | | | Stabilizer content in the formulation | | | | | | | | | | | |
| | | Accept- | Spray-dried powder | | | | Spray-dried powder | | | | Lyophilized | | | |
| | | ance | without PEG400 | | | | with PEG400 | | | | powder | | | |
| Test items | | criteria | 0 M | 3 M | 6 M | 9 M | 0 M | 3 M | 6 M | 9 M | 0 M | 3 M | 6 M | 9 M |
| pH | | 5.0-7.0 | 6.8 | 6.7 | 6.5 | 6.5 | 6.7 | 6.8 | 6.7 | 6.7 | 6.3 | 6.3 | 6.2 | 6.2 |
| Water (%) | | ≤2.5 | 2.4 | 2.4 | 2.5 | 2.7 | 1.6 | 1.6 | 1.6 | 1.6 | 0.8 | 0.8 | 0.8 | 1.0 |
| Reconstitution | | / | 3 | 3 | 1 | 1 | 2 | 2 | 2 | 1 | 2 | 3 | 2 | 2 |
| time (min) | | | | | | | | | | | | | | |
| Im- | Tazobactam | ≤0.3 | 0.08 | 0.10 | 0.13 | 0.18 | 0.05 | 0.05 | 0.07 | 0.10 | 0.09 | 0.09 | 0.09 | 0.10 |
| purities | related | | | | | | | | | | | | | |
| (%) | substance A | | | | | | | | | | | | | |
| | Amoxicillin | ≤0.2 | N.D. | N.D. | N.D. | N.D. | N.D. | N.D. | N.D. | N.D. | N.D. | N.D. | N.D. | N.D. |
| | related | | | | | | | | | | | | | |
| | substance A | | | | | | | | | | | | | |
| | Piperacillin | ≤0.8 | 0.18 | 0.20 | 0.23 | 0.23 | 0.11 | 0.12 | 0.13 | 0.15 | 0.14 | 0.17 | 0.18 | 0.20 |
| | related | | | | | | | | | | | | | |
| | substance E | | | | | | | | | | | | | |
| | Formyl | ≤0.2 | N.D. | N.D. | N.D. | N.D. | N.D. | N.D. | N.D. | N.D. | N.D | N.D. | N.D. | N.D. |
| | penicill- | | | | | | | | | | | | | |
| | amine | | | | | | | | | | | | | |
| | Ampicillin | ≤0.2 | 0.04 | 0.04 | 0.05 | 0.05 | N.D. | 0.02 | 0.02 | 0.02 | 0.04 | 0.04 | 0.04 | 0.05 |
| | Piper- | ≤0.2 | N.D. | N.D. | N.D. | N.D. | N.D. | N.D. | N.D. | N.D. | N.D. | N.D. | N.D. | N.D. |
| | azinedione- | | | | | | | | | | | | | |
| | carbonyl | | | | | | | | | | | | | |
| | D-phenyl- | | | | | | | | | | | | | |
| | glycyl- | | | | | | | | | | | | | |
| | penicillin | | | | | | | | | | | | | |
| | Acetylated | ≤0.5 | 0.03 | 0.05 | 0.02 | 0.07 | 0.03 | 0.05 | 0.02 | 0.04 | 0.04 | 0.06 | 0.03 | 0.03 |
| | piperacillin | | | | | | | | | | | | | |
| | penicilloic | | | | | | | | | | | | | |
| | acid | | | | | | | | | | | | | |
| | Piperacillin | ≤0.15 | 0.08 | N.D. | 0.10 | 0.20 | 0.07 | N.D. | 0.04 | 0.07 | 0.10 | 0.02 | 0.06 | 0.09 |
| | penicilloic | | | | | | | | | | | | | |
| | acid | | | | | | | | | | | | | |
| | isomer 1 | | | | | | | | | | | | | |
| | Piperacillin | ≤1.5 | 0.48 | 0.84 | 0.92 | 1.1 | 0.32 | 0.49 | 0.43 | 0.48 | 0.45 | 0.66 | 0.54 | 0.48 |
| | penicilloic | | | | | | | | | | | | | |
| | acid | | | | | | | | | | | | | |
| | isomer 2 | | | | | | | | | | | | | |
| | L-Piper- | — | N.D. | N.D | N.D. | N.D. | N.D. | N.D. | N.D. | N.D. | N.D. | N.D. | N.D. | N.D. |
| | acillin | | | | | | | | | | | | | |
| | Piperacillin | ≤0.5 | 0.05 | 0.10 | 0.17 | 0.25 | 0.04 | 0.07 | 0.13 | 0.13 | N.D. | 0.05 | 0.04 | 0.03 |
| | penilloic | | | | | | | | | | | | | |
| | acid | | | | | | | | | | | | | |
| | Piperacillin | — | 0.08 | 0.11 | 0.085 | 0.08 | 0.08 | 0.11 | 0.084 | 0.08 | N.D. | N.D. | N.D | N.D. |
| | methyl ester | | | | | | | | | | | | | |
| | Piperacillin | ≤0.2 | 0.03 | 0.04 | 0.04 | N.D. | 0.03 | 0.04 | 0.04 | N.D. | 0.04 | 0.04 | 0.04 | N.D. |
| | dimer | | | | | | | | | | | | | |
| | ethyl ester | | | | | | | | | | | | | |
| | Piperacillin | ≤0.2 | N.D. | N.D. | N.D. | 0.01 | N.D. | N.D. | N.D. | 0.03 | N.D. | N.D. | N.D. | 0.01 |
| | dimer | | | | | | | | | | | | | |
| | thiazol- | | | | | | | | | | | | | |
| | amide | | | | | | | | | | | | | |
| | derivative | | | | | | | | | | | | | |
| | Piperacillin | ≤0.3 | 0.02 | 0.02 | 0.01 | N.D | 0.02 | 0.02 | 0.02 | 0.02 | N.D. | N.D. | N.D. | N.D. |
| | penicill- | | | | | | | | | | | | | |
| | amide | | | | | | | | | | | | | |
| | Piperacillin | ≤0.4 | 0.04 | 0.06 | 0.07 | 0.02 | 0.04 | 0.07 | 0.07 | 0.02 | 0.10 | 0.11 | 0.13 | 0.02 |
| | dimer | | | | | | | | | | | | | |
| | Piper- | ≤0.3 | 0.33 | 0.32 | 0.32 | 0.08 | 0.33 | 0.33 | 0.33 | 0.08 | 0.22 | 0.22 | 0.22 | 0.12 |
| | acillinyl- | | | | | | | | | | | | | |
| | ampicillin | | | | | | | | | | | | | |
| | Individual | ≤0.1 | 0.10 | 0.09 | 0.06 | 0.34 | 0.10 | 0.09 | 0.06 | 0.38 | 0.07 | 0.10 | 0.07 | 0.24 |
| | impurity | | | | | | | | | | | | | |
| | Total | ≤4.0 | 1.84 | 2.37 | 2.46 | 3.0 | 1.49 | 1.78 | 1.67 | 2.0 | 1.55 | 1.84 | 1.61 | 1.7 |
| | impurities | | | | | | | | | | | | | |
| Assay | Piperacillin | 90.0~ | 103.5 | 100.8 | 100.4 | 96.8 | 103.3 | 100.8 | 100.5 | 96.3 | 102.3 | 100.3 | 100.0 | 100.4 |
| (%) | | 110.0 | | | | | | | | | | | | |
| | Tazobactam | 90.0~ | 103.0 | 102.6 | 102.0 | 96.1 | 102.8 | 102.6 | 102.1 | 95.0 | 101. | 101.5 | 100.4 | 99.8 |
| | | 110.0 | | | | | | | | | | | | |
| | Piperacillin: | 8:1 | 8:1 | 8:1 | 8:1 | 8:1 | 8:1 | 8:1 | 8:1 | 8:1 | 8:1 | 8:1 | 8:1 | 8:1 |
| | Tazobactam | | | | | | | | | | | | | |

Note:
Piperacillinylampicillin is a process-related impurity entrained in API, not a degradation impurity, and is stable or declining during the stability study.

As can be seen from Tables 8 and 9: (1) As compared with the spray-dried powder without a stabilizer, the piperacillin and tazobactam spray-dried powder with PEG has a lower water content, less impurities, and is more stable. (2) As compared with RLD lyophilized powder, the water content of piperacillin and tazobactam spray-dried powder with a PEG is slightly higher than the RLD freeze-dried powder due to different processes. However, their stability is comparable.

### Example 9: Effect of PEG 400 on the Biological Potency of Piperacillin and Tazobactam

As labeled in the Instruction for Use of piperacillin and tazobactam for injection, it has been verified in the study in viro and the study of clinical infections that piperacillin and tazobactam has the anti-bacterial activity against the following bacteria.

| | |
|---|---|
| Gram-positive bacteria | Staphylococcus aureus (methicillin |
| | susceptible isolates only) |
| Gram-negative bacteria | Acinetobacter baumannii |
| | Escherichia coli |
| | Haemophilus influenzae (excluding beta- |
| | lactamase negative, ampicillin-resistant |
| | isolates) |
| | Klebsiella pneumoniae |
| | Pseudomonas aeruginosa (administered in |
| | combination with an aminoglycoside to |
| | which the isolate is susceptible) |
| Anaerobic bacteria | Bacteroides spp. (Bacteroides fragilis, |
| | Bacteroides ovatus, Bacteroides |
| | thetaiotaomicron, Bacteroides vulgatus) |

Using Wyeth RLD (lot No. ALM9/11) as a control, the potency of piperacillin and tazobactam spray-dried powder with PEG 400 (batch No.: 200808-2) was assayed. The effect of PEG 400 on the potency of piperacillin and tazobactam spray-dried powder was studied by using Escherichia coli and Staphylococcus aureus as test bacteria, respectively.

9.1 Potency Assay by Using Escherichia coli as Test Bacterium

Methods: Cylinder Plate method.

Escherichia coli was used as the test bacterium at a concentration of 100U: 200U in a two-dose method. The inhibitory zone was between 18-22 cm. The reliability line rate and P-value were qualified.

9.2 Potency Assay by Using Staphylococcus aureus as Test Bacterium

Methods: Turbidimetric method.

A turbidimetric method was performed on Wyeth RLD at different gradient concentration units using Staphylococcus aureus to give a standard curve. The standard curve plotted according to 1.6 U, 2.0 U, 2.5 U, 3.125 U, 3.906 U concentration ratio has a R²=0.9908, indicating a linear result.

Therefore, the potency of piperacillin and tazobactam can be assayed by turbidimetric method in the concentration range of 1.6-3.9. Using Wyeth RLD as the standard, the biological potency of piperacillin and tazobactam spray-dried powder containing PEG 400 (batch No. 200808-2) was assayed by using Staphylococcus aureus as the test bacterium which were diluted to a high and a low doses of 3.2 U and 1.6 U, respectively.

The results of the above biological potency assay are shown in Table 10.

TABLE 10
The potency of piperacillin and tazobactam
Spray-dried powder as compared with RLD

| Test Bacterium | Sample | Batch No. | Potency | |
|---|---|---|---|---|
| Escherichia coli | RLD | ALM9/11 | 1000 | U/mg |
| | Spray-dried powder | 200808-2 | 999 | U/mg |
| Staphylococcus | RLD | ALM9/11 | 1000 | U/mg |
| aureus | Spray-dried powder | 200808-2 | 994 | U/mg |

It can be seen from Table 10 that the potency of piperacillin and tazobactam spray-dried powder containing PEG 400 is equivalent to the Wyeth RLD, indicating that PEG 400 has no influence on the antimicrobial activity of Piperacillin and Tazobactam,

### INDUSTRIAL APPLICABILITY

The method for industrial production of piperacillin and tazobactam spray-dried powder of the present invention can produce high-purity piperacillin and tazobactam powder with high efficiency and high yield, and has a high application value in the pharmaceutical field.

1. A method for industrial production of piperacillin and tazobactam spray-dried powder, comprising the steps of:
(1) preparing a mixture solution of piperacillin or a pharmaceutically acceptable salt thereof and tazobactam or a pharmaceutically acceptable salt thereof while maintaining the pH value at 6.0~7.5;
(2) adding a stabilizer to the solution obtained in the step (1) to give a mixed solution, wherein the stabilizer is one or more selected from the group consisting of carbohydrates, amino acids and polyols; and
(3) spray drying the mixed solution obtained in the step (2) to give the piperacillin and tazobactam spray-dried powder.

2. The method for industrial production of piperacillin and tazobactam spray-dried powder according to claim 1, characterized in that:
in the mixture solution prepared by the step (1), the ratio of the content of piperacillin sodium or a pharmaceutically acceptable salt thereof calculated as piperacillin free acid to the content of tazobactam or a pharmaceutically acceptable salt thereof calculated as tazobactam free acid is approximately 8:1.

3. The method for industrial production of piperacillin and tazobactam spray-dried powder according to claim 1, characterized in that:
in the step (1), a powder mixture of piperacillin or a pharmaceutically acceptable salt thereof and tazobactam or a pharmaceutically acceptable salt thereof is dissolved in water to prepare the mixture solution; alternatively, piperacillin or a pharmaceutically acceptable salt thereof and tazobactam or a pharmaceutically acceptable salt thereof are dissolved in water, separately, and then mixed to prepare the mixture solution; alternatively, piperacillin acid and/or tazobactam acid is converted into a salt with sodium bicarbonate, sodium carbonate or sodium hydroxide to prepare a solution of piperacillin sodium and/or tazobactam sodium, respectively, and then formulated into the mixture solution.

4. The method for industrial production of piperacillin and tazobactam spray-dried powder according to claim 1, characterized in that:
the concentration calculated as piperacillin of the mixed solution obtained in the step (2) is 1~50% by weight.

5. The method for industrial production of piperacillin and tazobactam spray-dried powder according to claim 1, characterized in that:
the concentration calculated as piperacillin of the mixed solution obtained in the step (2) is 26~38% by weight.

6. The method for industrial production of piperacillin and tazobactam spray-dried powder according to claim 1, characterized in that:
the stabilizer added in the step (2) is a polyol.

7. The method for industrial production of piperacillin and tazobactam spray-dried powder according to claim 6, characterized in that:
the stabilizer is polyethylene glycol or glycerol.

8. The method for industrial production of piperacillin and tazobactam spray-dried powder according to claim 6, characterized in that:
the stabilizer is polyethylene glycol 200~600.

9. The method for industrial production of piperacillin and tazobactam spray-dried powder according to claim 6, characterized in that:
the stabilizer is polyethylene glycol 300~400.

10. The method for industrial production of piperacillin and tazobactam spray-dried powder according to claim 1, characterized in that:
in the step (2), 0~50 parts by weight of the stabilizer is added based on 100 parts by weight of piperacillin.

11. The method for industrial production of piperacillin and tazobactam spray-dried powder according to claim 1, characterized in that:
in the step (2), 0~20 parts by weight of the stabilizer is added based on 100 parts by weight of piperacillin.

12. The method for industrial production of piperacillin and tazobactam spray-dried powder according to claim 1, characterized in that:
in the step (2), 2.5~15 parts by weight of the stabilizer is added based on 100 parts by weight of piperacillin.

13. The method for industrial production of piperacillin and tazobactam spray-dried powder according to claim 1, characterized in that:
in the step (2), an aminocarboxylic acid chelating agent EDTA is further added to the solution obtained in the step (1).

14. The method for industrial production of piperacillin and tazobactam spray-dried powder according to claim 1, characterized in that:
the mixed solution obtained in the step (2) is stored at a storage temperature of −12~8° C.

15. The method for industrial production of piperacillin and tazobactam spray-dried powder according to claim 1, characterized in that:
the mixed solution obtained in the step (2) is stored at a storage temperature of −10~0° C.

16. The method for industrial production of piperacillin and tazobactam spray-dried powder according to claim 1, characterized in that:
in the step (3), the spray drying is carried out at an inlet air temperature of 160~240° C.

17. The method for industrial production of piperacillin and tazobactam spray-dried powder according to claim 1, characterized in that:
in the step (3), the spray drying is carried out at an inlet air temperature of 185~200° C.

18. The method for industrial production of piperacillin and tazobactam spray-dried powder according to claim 1, characterized in that:
in the step (3), the spray drying is carried out at an outlet air temperature of 80~130° C.

19. The method for industrial production of piperacillin and tazobactam spray-dried powder according to claim 1, characterized in that:
in the step (3), the spray drying is carried out at an outlet air temperature of 100~115° C.

20. A piperacillin and tazobactam spray-dried powder obtainable by the method according to claim 1, wherein piperacillin and tazobactam spray-dried powder comprises piperacillin or a pharmaceutically acceptable salt thereof, tazobactam or a pharmaceutically acceptable salt thereof and one or more stabilizers selected from the group consisting of carbohydrates, amino acids and polyols.

21. The piperacillin and tazobactam spray-dried powder according to claim 20, wherein the stabilizer is a polyol.

22. The piperacillin and tazobactam spray-dried powder according to claim 20, wherein the stabilizer is polyethylene glycol or glycerol.

23. The piperacillin and tazobactam spray-dried powder according to claim 20, wherein the stabilizer is polyethylene glycol 200~600.

24. The piperacillin and tazobactam spray-dried powder according to claim 20, wherein the stabilizer is polyethylene glycol 300~400.

25. The piperacillin and tazobactam spray-dried powder according to claim 20, wherein the stabilizer is contained in an amount of 0~50 parts by weight based on 100 parts by weight of piperacillin.

26. The piperacillin and tazobactam spray-dried powder according to claim 20, wherein the stabilizer is contained in an amount of 0~20 parts by weight based on 100 parts by weight of piperacillin.

27. The piperacillin and tazobactam spray-dried powder according to claim 20, wherein the stabilizer is contained in an amount of 2.5~15 parts by weight based on 100 parts by weight of piperacillin.
